(19) United States
(12) Patent Application Publication
(10) Pub. No.: US 2013/0171105 A1
(43) Pub. Date: Jul. 4, 2013

(54) CANCER THERAPY USING A COMBINATION OF A HSP90 INHIBITORY COMPOUND AND A TOPOISOMERASE II INHIBITOR

(71) Applicants: Ronald K. Blackman, Brookline, MA (US); Kevin Paul Foley, Landsdale, PA (US); David Proia, Newton, MA (US)

(72) Inventors: Ronald K. Blackman, Brookline, MA (US); Kevin Paul Foley, Landsdale, PA (US); David Proia, Newton, MA (US)

(73) Assignee: SYNTA PHARMACEUTICALS CORP., Lexington, MA (US)

(21) Appl. No.: 13/699,517

(22) PCT Filed: May 23, 2011
(86) PCT No.: PCT/US11/37529

§ 371 Date: Mar. 7, 2013

Related U.S. Application Data
(60) Provisional application No. 61/347,685, filed on May 24, 2010.

(51) Int. Cl. A61K 31/4196; A61K 31/7048; A61K 39/395; A61K 38/21; A61K 31/704; A61K 31/568; A61K 33/24; A61K 31/69; A61K 31/65; A61K 31/555; A61K 31/664; A61K 38/19; A61K 31/675; A61K 31/439

(52) U.S. Cl. CPC A61K 31/4196; A61K 31/675; A61K 31/7048; A61K 39/3955; A61K 38/212; A61K 31/704; A61K 31/439; A61K 33/24; A61K 31/69; A61K 31/65; A61K 31/555; A61K 31/664; A61K 38/193; A61K 31/568
USPC 424/85.7; 514/93; 514/384; 514/27; 514/383; 424/133.1; 424/649; 424/85.1

(57) ABSTRACT

A pharmaceutical combination comprising a topoisomerase II inhibitor, and an Hsp90 inhibitor according to the following formulae a tautomer, or a pharmaceutically acceptable salt thereof, wherein the variables in the structural formulae are defined herein. Also provided is a method for treating a proliferative disorder in a subject in need thereof, using the pharmaceutical combination described herein.

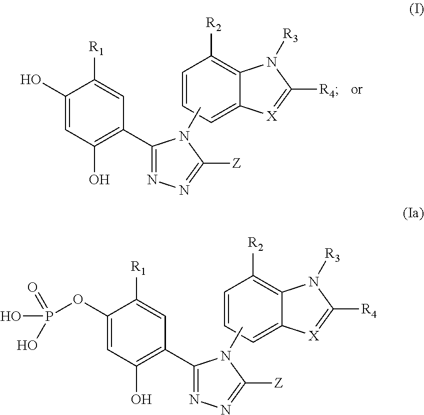

### CROSS-REFERENCE TO RELATED PATENT APPLICATIONS

This application claims the benefit of U.S. Provisional Application No. 61/347,685, filed on May 24, 2010, the entire disclosure of which is incorporated herein by reference.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that certain triazolone Hsp90 inhibitors and topoisomerase II inhibitor combinations are surprisingly effective at treating subjects with certain cancers without further increasing the side effect profile of the single agents. The particular combination therapies disclosed herein demonstrate surprising biological activity by demonstrating significant anticancer effects.

The present method utilizes Hsp90 inhibitors according to formulae (I) or (Ia), or a compound in Tables 1 or 2 for the treatment of proliferative disorders, such as cancer, in combination with a topoisomerase II inhibitor. The method of treating a subject with cancer includes the step of administering to the subject an Hsp90 inhibitor according to formulae (I) or (Ia), or a compound in Tables 1 or 2 and a topoisomerase II inhibitor useful for the treatment of cancer. In one embodiment, the administration of the Hsp90 inhibitor and the topoisomerase II inhibitor are done concurrently. In another embodiment, the administration of the Hsp90 inhibitor and the topoisomerase II inhibitor are done sequentially. In another embodiment, the administration of the Hsp90 inhibitor and the topoisomerase II inhibitor are dosed independently. In any one of these embodiments, the topoisomerase II inhibitor may be etoposide, HU-331, ICRF-187 (dexrazoxane), ICRF-193, amsacrine, mitindomide, teniposide, doxorubicin, daunorubicin, idarubicin, mitoxantrone, anteniposide or novobiocin. In any one of these embodiments, the Hsp90 inhibitor is a compound represented in Tables 1 or 2. In any one of these embodiments, the topoisomerase II inhibitor may be etoposide.

In one embodiment, the cancer has mutations or translocations in EGFR, K-ras, HER2neu, B-raf, PI3K and/or ALK proteins. In one embodiment, the cancer has wild type EGFR and K-ras. In one embodiment, the cancer has mutations in EGFR and wild type K-ras. In one embodiment, the cancer has wild type EGFR and mutations in the K-ras protein. In one embodiment, the cancer has the ALK-elm4 translocation. In one embodiment, the cancer has the HER2neu mutation. In one embodiment, the cancer has a mutation in PI3K. In one embodiment, the cancer has a mutation in the B-raf protein.

The present invention also includes a kit for administration of the combination therapy. In one embodiment, the kit includes separate pharmaceutical compositions containing the Hsp90 inhibitor according to formulae (I) or (Ia) or a compound in Tables 1 or 2, and the topoisomerase II inhibitor. In another embodiment, the kit includes one pharmaceutical composition containing both the Hsp90 inhibitor and the topoisomerase II inhibitor. In any of these embodiments, each pharmaceutical composition includes one or more pharmaceutically acceptable carrier or diluent. In any one of these embodiments, the topoisomerase II inhibitor may be etoposide, HU-331, ICRF-187 (dexrazoxane), ICRF-193, amsacrine, mitindomide, teniposide, doxorubicin, daunorubicin, idarubicin, mitoxantrone, anteniposide or novobiocin. In any one of these embodiments, the Hsp90 inhibitor may be a compound represented in Tables 1 or 2. In any one of these embodiments, the topoisomerase II inhibitor may be etoposide.

In one embodiment, the invention includes use of an Hsp90 inhibitor according to formulae (I) or (Ia) or a compound in Tables 1 or 2 for the manufacture of a medicament for treating cancer in combination with a topoisomerase II inhibitor.

In one embodiment, the method includes the treatment of drug-resistant cancer in a subject by administering an effective amount of the pharmaceutical combination comprising an Hsp90 compound according to formulae (I) or (Ia) or a compound in Tables 1 or 2 and a topoisomerase II inhibitor. In one embodiment, the method further comprises the administration of one or more therapeutic agents in addition to the pharmaceutical combination of an Hsp90 compound according to formulae (I) or (Ia) or a compound in Tables 1 or 2 and a topoisomerase II inhibitor. In certain embodiments, the combination treatment utilizing an Hsp90 compound according to formulae (I) or (Ia) or a compound in Tables 1 or 2 with a topoisomerase II inhibitor to help to arrest, partially or fully, or reduce the development of drug resistant cancer in a subject. In this embodiment, the combinations described herein may allow a reduced dose of the topoisomerase II inhibitor given to a subject, because the Hsp90 inhibitor should inhibit the development of multidrug resistant cancerous cells. In one embodiment, the topoisomerase II inhibitor may be etoposide, HU-331, ICRF-187 (dexrazoxane), ICRF-193, amsacrine, mitindomide, teniposide, doxorubicin, daunorubicin, idarubicin, mitoxantrone, anteniposide or novobiocin. In another embodiment, the topoisomerase II inhibitor may be etoposide.

### BRIEF DESCRIPTION OF THE DRAWINGS

The foregoing and other objects, features and advantages of the invention will be apparent from the following more particular description of some embodiments of the invention, as illustrated in the accompanying drawings in which like reference characters refer to the same parts throughout the different views. The drawings are not necessarily to scale, emphasis instead being placed upon illustrating the principles of the invention.

FIG. 1A showed a dose-dependent curve with the IC₅₀ of compound 1 being indicated.

FIG. 1B showed a dose-dependent curve with the IC₅₀ of etoposide being indicated.

FIG. 2 showed the percent of K562 cells that were killed by Compound 1, etoposide or the combination of the two drugs at the indicated concentrations.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker Alkoxy groups included in compounds described herein may be optionally substituted with one or more substituents.

As used herein, a “haloalkoxy” is a haloalkyl group which is attached to another moiety via an oxygen linker

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR″, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³° or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C₁-C₄ alkyl, C₁-C₄ haloalkyl, C₁-C₄ alkoxy, C₁-C₄ haloalkoxy, C₁-C₄ hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

As used herein, the term “compound(s) described herein” or similar terms refers to a compound of formulae (I), or (Ia) or a compound in Tables 1 or 2 or a tautomer or pharmaceutically acceptable salt thereof. Also included in the scope of the embodiments are a solvate, the anhydrous form, clathrate, non-clathrate, hydrate, amorphous or polymorph of a compound of formulae (I), or (Ia), or a compound in Tables 1 or 2.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water. Also included in the anhydrous form of the compound, which, as the term is used herein, refers to crystalline forms of the compounds in which substantially no solvent is incorporated into the crystal lattice.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water). Also included is the non-clathrate, which refers to the compound in the form of a crystal lattice without guest molecules trapped within.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

DNA topoisomerases are enzymes present in all cells that catalyze topological changes in DNA. Topoisomerase II (“topo II”) plays important roles in DNA replication, chromosome segregation and the maintenance of the nuclear scaffold in eukaryotic cells. The enzyme acts by creating breaks in DNA, thereby allowing the DNA strands to unravel and separate. Due to the important roles of the enzyme in dividing cells, the enzyme is a highly attractive target for chemotherapeutic agents, especially in human cancers. Topo II and Hsp90 belong to a small group of proteins that share the same ATP binding domain known as the Bergerat fold. It has been reported that the Hsp90 inhibitor, radicicol, can inhibit the activity of human topo II, most likely by interacting with the ATP-binding site of the enzyme. Gadelle, D., et al., Biochemical Pharmacology, 72 (10), 1207, 2006.

Some of the currently known Topo II inhibitors include etoposide, amsacrine, mitindomide, teniposide, doxorubicin, daunorubicin, idarubicin, mitoxantrone, anteniposide, novobiocin, HU-331 (3S,4R-p-benzoquinone-3-hydroxy-2-p-mentha-(1,8)-dien-3-yl-5-pentyl), ICRF-187 (dexrazoxane), and ICRF-193 (4-[2-(3,5-dioxo-1-piperazinyl)-1-methylpropyl]piperazine-2,6-dione). Etoposide, alone or in combination, is currently being tested in clinical trials for non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, or AID's related Karposi's sarcoma. Etoposide is approved in the United States to treat refractory testicular cancer and small cell lung cancer. Topoisomerase II inhibitors are effective in hematological cancer in addition to solid tumors, and are often administered to pediatric patients with various types of leukemia.

“Topoisomerase II inhibitors” used herein include compounds that classically inhibit topoisomerase II, and those compounds that may be characterized as “topoisomerase II poisons”, which target the DNA-protein complex. The topoisomerase II inhibitors employed herein include etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, some of which are all approved anti-cancer drugs which act by disrupting the DNA unwinding/replication process in the cell cycle. Inhibition of topoisomerase II inhibits a cell's reentry into the cell division cycle (G1) from the quiescent (GO) state.

The term “c-Kit” or “c-Kit kinase” refers to a membrane receptor protein tyrosine kinase which is preferably activated upon binding Stem Cell Factor (SCF) to its extracellular domain. Yarden, et al., Embo. J., (1987)11:3341-3351; Qiu, et al., Embo. J., (1988) 7:1003-1011. The full length amino acid sequence of a c-Kit kinase preferably is as set forth in Yarden, et al.; and Qiu, et al., which are incorporated by reference herein in their entirety, including any drawings. Mutant versions of c-Kit kinase are encompassed by the term “c-Kit” or “c-Kit kinase” and include those that fall into two classes: (1) having a single amino acid substitution at codon 816 of the human c-Kit kinase, or its equivalent position in other species (Ma, et al., J. Invest Dermatol., (1999) 112:165-170), and (2) those which have mutations involving the putative juxtamembrane z-helix of the protein (Ma, et al., J. Biol. Chem., (1999) 274:13399-13402). Both of these publications are incorporated by reference herein in their entirety, including any drawings.

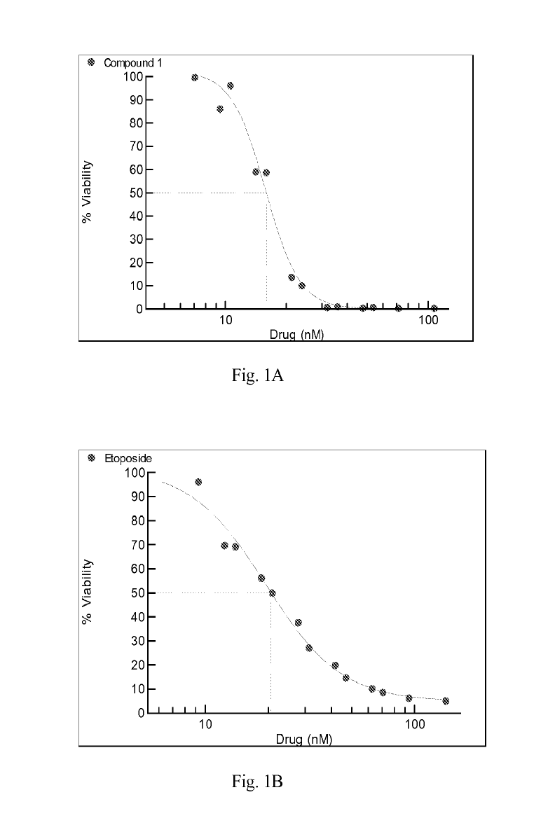

As used herein, “BCR-ABL” is a fusion protein that results from the translocation of gene sequences from c-ABL protein tyrosine kinase on chromosome 9 into BCR sequences on chromosome 22 producing the Philadelphia chromosome. A schematic representation of human BCR, ABL and BCR-ABL can be seen in FIG. 1 of U.S. patent application Ser. No. 10/193,651, filed on Jul. 9, 2002. Depending on the breaking point in the BCR gene, BCR-ABL fusion proteins can vary in size from 185-230 kDa but they must contain at least the OLI domain from BCR and the TK domain from ABL for transforming activity. The most common BCR-ABL gene products found in humans are P230 BCR-ABL, P210 BCR-ABL and P190 BCR-ABL. P210 BCR-ABL is characteristic of CML and P190 BCR-ABL is characteristic of ALL.

FLT3 kinase is a tyrosine kinase receptor involved in the regulation and stimulation of cellular proliferation. Gilliland, et al., Blood (2002), 100:1532-42. The FLT3 kinase has five immunoglobulin-like domains in its extracellular region, as well as an insert region of 75-100 amino acids in the middle of its cytoplasmic domain. FLT3 kinase is activated upon the binding of the FLT3 ligand which causes receptor dimerization. Dimerization of the FLT3 kinase by FLT3 ligand activates the intracellular kinase activity as well as a cascade of downstream substrates including Stat5, Ras, phosphatidylinositol-3-kinase (PI3K), Erk2, Akt, MAPK, SHC, SHP2 and SHIP. Rosnet, et al., Acta Haematol. (1996), 95:218; Hayakawa, et al., Oncogene (2000), 19:624; Mizuki, et al., Blood (2000), 96:3907; Gilliand, et al., Curr. Opin. Hematol. (2002), 9: 274-81. Both membrane-bound and soluble FLT3 ligand bind, dimerize, and subsequently activate the FLT3 kinase.

Normal cells that express FLT3 kinase include immature hematopoietic cells, typically CD34+ cells, placenta, gonads and brain. Rosnet, et al., Blood (1993), 82:1110-19; Small, et al., Proc. Natl. Acad. Sci. U.S.A. (1994), 91:459-63; Rosnet, et al., Leukemia (1996), 10:238-48. However, efficient stimulation of proliferation via FLT3 kinase typically requires other hematopoietic growth factors or interleukins FLT3 kinase also plays a critical role in immune function through its regulation of dendritic cell proliferation and differentiation. McKenna, et al., Blood (2000), 95:3489-497. Numerous hematologic malignancies express FLT3 kinase, the most prominent of which is AML. Yokota, et al., Leukemia (1997), 11:1605-09. Other FLT3 expressing malignancies include B-precursor cell acute lymphoblastic leukemias, myelodysplastic leukemias, T-cell acute lymphoblastic leukemias, and chronic myelogenous leukemias. Rasko, et al., Leukemia (1995), 9:2058-66.

FLT3 kinase mutations associated with hematologic malignancies are activating mutations. In other words, the FLT3 kinase is constitutively activated without the need for binding and dimerization by FLT3 ligand, and therefore stimulates the cell to grow continuously. Two types of activating mutations have been identified: internal tandem duplications (ITDs) and point mutation in the activating loop of the kinase domain. As used herein, the term “FLT3 kinase” refers to both wild type FLT3 kinase and mutant FLT3 kinases, such as FLT3 kinases that have activating mutations. Compounds provided herein are useful in treating conditions characterized by inappropriate FLT3 activity, such as proliferative disorders. Inappropriate FLT3 activity includes, but is not limited to, enhanced FLT3 activity resulting from increased or de novo expression of FLT3 in cells, increased FLT3 expression or activity and FLT3 mutations resulting in constitutive activation. The existence of inappropriate or abnormal FLT3 ligand and FLT3 levels or activity can be determined using well known methods in the art. For example, abnormally high FLT3 levels can be determined using commercially available ELISA kits. FLT3 levels can also be determined using flow cytometric analysis, immunohistochemical analysis and in situ hybridization techniques.

“Epidermal growth factor receptor” or “EGFR”, as used herein, means any epidermal growth factor receptor (EGFR) protein, peptide, or polypeptide having EGFR or EGFR family activity (e.g., Her1, Her2, Her3 and/or Her4), such as encoded by EGFR Genbank Accession Nos. shown in Table I of U.S. patent application Ser. No. 10/923,354, filed on Aug. 20, 2004, or any other EGFR transcript derived from a EGFR gene and/or generated by EGFR translocation. The term “EGFR” is also meant to include other EGFR protein, peptide, or polypeptide derived from EGFR isoforms (e.g., Her1, Her2, Her3 and/or Her4), mutant EGFR genes, splice variants of EGFR genes, and EGFR gene polymorphisms.

EGFR is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor a (TGFa), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands. Gullick, Br. Med. Bull. (1991), 47:87-98; Modijtahedi & Dean, Int. J. Oncol. (1994), 4:277-96; Salomon, et al., Crit. Rev. Oncol. Hematol. (1995), 19:183-232. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Lei, et al., Anti-cancer Res. (1999), 19:221-28; Veale, et al., Br. J. Cancer (1993); 68:162-65. Mutations in EGFR are associated with many types of cancer as well. For example, EGFR mutations are highly prevalent in non-mucinous BAC patients. Finberg, et al., J. Mol. Diagnostics (2007) 9(3):320-26.

As used herein, the term “c-Kit associated cancer” refers to a cancer which has aberrant expression and/or activation of c-Kit. c-Kit associated cancers include leukemias, mast cell tumors, small cell lung cancer, testicular cancer, some cancers of the gastrointestinal tract and some cancers of the central nervous system. In addition, c-Kit has been implicated in playing a role in carcinogenesis of the female genital tract (Inoue, et al., Cancer Res., (1994) 54(11):3049-3053), sarcomas of neuroectodermal origin (Ricotti, et al., Blood, (1998) 91:2397-2405), and Schwann cell neoplasia associated with neurofibromatosis (Ryan, et al., J. Neuro. Res., (1994) 37:415-432).

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy, (e.g., diabetic retinopathy or other retinopathies), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis and desmoid tumors. Non-cancerous proliferative disorders also include hyperproliferation of cells in the skin such as psoriasis and its varied clinical forms, Reiter's syndrome, pityriasis rubra pilaris, hyperproliferative variants of disorders of keratinization (e.g., actinic keratosis, senile keratosis), scleroderma, and the like. In one embodiment, the proliferative disorder is cancer.

One embodiment of the invention is a method of treating a proliferative disorder in a subject, comprising administering to the subject an effective amount of the combination of Hsp90 inhibitor and topoisomerase II inhibitor as described herein. In one embodiment, the proliferative disorder is cancer. In one embodiment, the cancer is selected from non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma. In one embodiment, the cancer is selected from refractory testicular cancer, small cell lung cancer, Hodgkin's disease, glioblastoma multiforme and ovarian cancer. In one embodiment, the cancer is small cell lung cancer. In one embodiment, the cancer is refractory testicular cancer. In one embodiment, the cancer is non-small cell lung cancer. In one embodiment, the cancer is glioblastoma multiforme. In one embodiment, the cancer is ovarian cancer. In one embodiment, the cancer has a mutation or translocation in EGFR, K-ras, PI3K, ALK, HER2neu and/or B-raf proteins.

In one embodiment, the method is used to treat leukemias, including acute and/or chronic leukemias, e.g., lymphocytic leukemia, e.g., as exemplified by the p388 (murine) cell line, large granular lymphocytic leukemia, and lymphoblastic leukemia; T-cell leukemias, e.g., T-cell leukemia, as exemplified by the CEM, Jurkat, and HSB-2 (acute), YAC-1 (murine) cell lines, T-lymphocytic leukemia, and T-lymphoblastic leukemia; B-cell leukemia, e.g., as exemplified by the SB (acute) cell line, and B-lymphocytic leukemia; mixed cell leukemias, e.g., B- and T-cell leukemia and B- and T-lymphocytic leukemia; myeloid leukemias, e.g., granulocytic leukemia, myelocytic leukemia, e.g., as exemplified by the HL-60 (promyelocyte) cell line, and myelogenous leukemia, e.g., as exemplified by the K562 (chronic)cell line; neutrophilic leukemia; eosinophilic leukemia; monocytic leukemia, e.g., as exemplified by the THP-1 (acute) cell line; myelomonocytic leukemia; Naegeli-type myeloid leukemia; and nonlymphocytic leukemia. Other examples of leukemias are described in Chapter 60 of THE CHEMOTHERAPY SOURCEBOOK (Michael C. Perry Ed., Williams & Williams (1992)) and Section 36 of HOLLAND FRIE CANCER MEDICINE (Bast et al. Eds., 5th ed., B.C. Decker Inc. (2000)).

In one embodiment, the disclosed method is believed to be effective in treating a subject with non-Hodgkin's lymphoma (NHL). Lymphomas are generally classified as either Hodgkin's disease (HD) or non-Hodgkin's lymphomas. NHL differs from HD by the absence of Reed-Sternberg cells. The course of NHL is less predictable than HD and is more likely to spread to areas beyond the lymph nodes. NHL can be further divided into B-cell NHL and T-cell NHL, each of which can be further categorized into a variety of different subtypes. For example, B-cell NHL includes Burkitt's lymphoma, follicular lymphoma, diffuse large B-cell lymphoma, nodal marginal zone B-cell lymphoma, plasma cell neoplasms, small lymphocytic lymphoma/chronic lymphocytic leukemia, mantle cell lymphoma, extranodal marginal zone B-cell lymphoma and lymphoplamacytic lymphoma/Waldenstrom macroglobulinemia. T-cell NHL includes anaplastic large-cell lymphoma, precursor-T-cell lymphoblastic leukemia/lymphoma, unspecified peripheral T-cell lymphoma, acute lymphoblastic leukemia/lymphoma, angioimmunoblastic T-cell lymphoma and mycosis fungoides. In one embodiment, the method is used to treat a subject with Hodgkin's Disease.

Some of the disclosed methods can be particularly effective at treating subjects whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Other anti-proliferative or anti-cancer therapies may be combined with the pharmaceutical combination of this invention to treat proliferative diseases and cancer. Other therapies or anti-cancer agents that may be used in combination with the inventive anti-cancer agents of the present invention include surgery, radiotherapy (including, but not limited to, gamma-radiation, neutron beam radiotherapy, electron beam radiotherapy, proton therapy, brachytherapy, and systemic radioactive isotopes), endocrine therapy, biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs. In one embodiment, the pharmaceutical combination of the invention is administered with one or more therapeutic agent selected from DFMO, vandetanib, trastuzumab, temodar, irinotecan, dexamethasone, cisplatin, epirubicin, ifosfamide, oxaliplatin, mitoxantrone, vorinostat, carboplatin, interferon alpha, rituximab, prednisone, cyclophosphamide, bendamustine, adriamycin, valproate, celecoxib, thalidomide, nelarabine, methotrexate, filgrastim, gemtuzumab ozogamicin, testosterone, clofarabine, cytarabine, everolimus, rituxumab, busulfan, capecitabine, pegfilgrastim, mesna, amrubicin, obatoclax, gefitinib, cyclosporine, dasatinib, temozolomide, thiotepa, plerixafor, mitotane, vincristine, doxorubicin, cixutumumab, endostar, fenofibrate, melphalan, sunitinib, rubitecan, enoxaparin, isotretinoin, tariquidar, pomalidomide, sorafenib, altretamine, idarubicin, rapamycin, zevalin, everolimus, pravastatin, carmustine, nelfinavir, streptozocin, tirapazamine, aprepitant, lenalidomide, G-CSF, procarbazine, alemtuzumab, amifostine, valspodar, lomustine, oblimersen, temsirolimus, vinblastine, figitumumab, belinostat, niacinamide, tipifarnib, estramustine, erlotinib, bevacizumab, paclitaxel, docetaxel, cisplatin, carboplatin, Abraxane®, pemetrexed, bortezomib, topotecan, cetuximab, gemcitabine and tetracycline. In one embodiment, the one or more therapeutic agent is selected from carboplatin, cisplatin, erlotinib, bevacizumab, bortezomib, paclitaxel, doxorubicin, docetaxel, mitoxantrone, cytarabine and vincristine

As used herein, the term “pharmaceutically acceptable salt” refers to a salt prepared from a compound of formulae (I) or (Ia) or a compound in Tables 1 or 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I) or (Ia) or a compound in Tables 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I) or (Ia) or a compound in Tables 1 or 2. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound described herein are provided herein below. In a specific embodiment, the invention provides a method of treating, managing, or ameliorating a disease or disorder, e.g. a proliferative disorder, or one or more symptoms thereof, the method comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month. Other suitable dosing regimens are disclosed in U.S. Provisional Application Ser. No. 61/484,988, filed May 11, 2011, entitled DOSING REGIMENS FOR TREATING CANCER WITH AN HSP90 INHIBITORY COMPOUND, the entire teachings of which are incorporated herein by reference.

The dosage of an individual topoisomerase II inhibitor used in the pharmaceutical combination may be equal to or lower than the dose of an individual therapeutic agent when given independently to treat, manage, or ameliorate a disease or disorder, or one or more symptoms thereof. In one embodiment of the invention, the disease or disorder being treated with a combination therapy is a proliferative disorder. In one embodiment, the proliferative disorder is cancer. In one embodiment, the topoisomerase II inhibitor etoposide is administered at a dose of between about 35 mg/m2 to about 100 mg/m2 by IV or orally once a day for 1, 2, 3, 4 or 5 days continuously per treatment cycle. In one embodiment, the etoposide is administered daily for 21 days. In one embodiment, the etoposide is administered at a dose between 60 and 250 mg/m2 per day for the length of the treatment in a particular cycle. A treatment cycle can last between one and 6 weeks. The recommended dosages of therapeutic agents currently used for the treatment, management, or amelioration of a disease or disorder, or one or more symptoms thereof, can obtained from any reference in the art. For a more in depth review of dosage and treatment schedules for various disorders, see, e.g., GOODMAN & GILMAN'S THE PHARMACOLOGICAL BASIS OF BASIS OF THERAPEUTICS 9^{Th} ED, (Hardman, et al., Eds., NY:McGraw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the topoisomerase II inhibitor are dosed on independent schedules. In another embodiment, the Hsp90 inhibitor and the topoisomerase II inhibitor are dosed on approximately the same schedule. In another embodiment, the Hsp90 inhibitor and the topoisomerase II inhibitor are dosed concurrently or sequentially on the same day.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a “racemic mixture” means about 50% of one enantiomer and about 50% of is corresponding enantiomer of the molecule. The combination encompasses all enantiomerically-pure, enantiomerically-enriched, diastereomerically pure, diastereomerically enriched, and racemic mixtures of the compounds described herein. Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

The methods described herein utilize triazolone compounds listed in Tables 1 or 2, or a compound represented by Formulae (I) or (Ia):

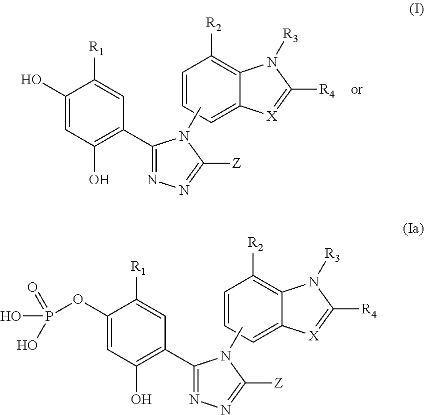

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ is selected from the group consisting of —H, lower alkyl, lower alkoxy, lower cycloalkyl, and lower cycloalkoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy.

In another embodiment, in formula (I) or (Ia), R₃ is selected from the group consisting of H, a lower alkyl, a lower cycloalkyl, —C(O)N(R₂₇)₂, and —C(O)OH, wherein R₂₇ is —H or a lower alkyl.

In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, R₄ is H or a lower alkyl.

In another embodiment, in formula (I) or (Ia), R₄ is selected from the group consisting of —H, methyl, ethyl, propyl, isopropyl or cyclopropyl.

In another embodiment, in formula (I) or (Ia), R₁ is selected from the group consisting of —H, —OH, —SH, —NH₂, a lower alkoxy and a lower alkyl amino.

In another embodiment, in formula (I) or (Ia), R₁ is selected from the group consisting of —H, —OH, methoxy and ethoxy.

In another embodiment, in formula (I) or (Ia), Z is —OH.

In another embodiment, in formula (I) or (Ia), Z is —SH.

In another embodiment, in formula (I) or (Ia), R₂ is selected from the group consisting of —H, —OH, —SH, —NH₂, a lower alkoxy and a lower alkyl amino.

In another embodiment, in formula (I) or (Ia), R₂ is selected from the group consisting of —H, —OH, methoxy, and ethoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH.

In another embodiment, the compound is selected from the group consisting of:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, the compound is selected from the group consisting of
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCL salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole,
3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

In another embodiment, the compound is selected from the group consisting of
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate, or a tautomer, or a pharmaceutically acceptable salt thereof.

Hsp90 inhibitory compounds, as well as tautomers or pharmaceutically acceptable salts thereof, that may be used in the methods described herein are depicted in Tables 1 or 2.

TABLE 1

| | STRUCTURE | TAUTOMERIC STRUCTURE | NAME |
|---|---|---|---|
| 1 | 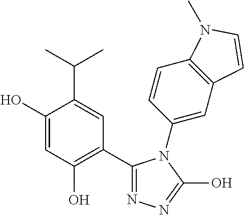 [SMILES: CC(C)c1cc(-c2nnc(O)n2-c2ccc3c(ccn3C)c2)c(O)cc1O] | 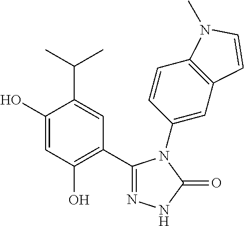 [SMILES: CC(C)c1cc(-c2n[nH]c(=O)n2-c2ccc3c(ccn3C)c2)c(O)cc1O] | 3-(2,4-DIHYDROXY-5- ISOPROPYL-PHENYL)-4-(1- METHYL-INDOL-5-YL)-5- HYDROXY-[1,2,4] TRIAZOLE |
| 2 | 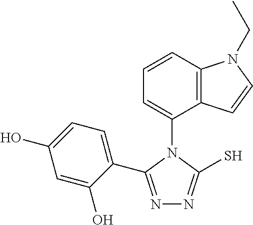 [SMILES: CCn1ccc2c(-n3c(S)nnc3-c3ccc(O)cc3O)cccc21] | 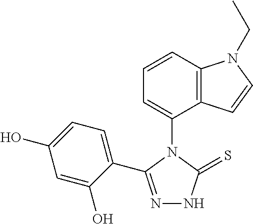 [SMILES: CCn1ccc2c(-n3c(-c4ccc(O)cc4O)n[nH]c3=S)cccc21] | 3-(2,4-DIHYDROXYPHENYL)- 4-(1-ETHYL-INDOL-4-YL)-5- MERCAPTO-[1,2,4] TRIAZOLE |
| 3 | 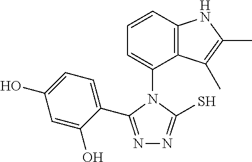 [SMILES: Cc1[nH]c2cccc(-n3c(S)nnc3-c3ccc(O)cc3O)c2c1C] | 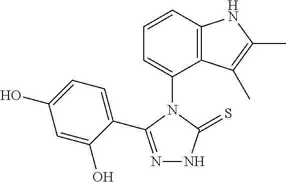 [SMILES: Cc1[nH]c2cccc(-n3c(-c4ccc(O)cc4O)n[nH]c3=S)c2c1C] | 3-(2,4-DIHYDROXY- PHENYL)-4-(2,3-DIMETHYL- 1H-INDOL-4-YL)-5- MERCAPTO-[1,2,4] TRIAZOLE |
| 4 | 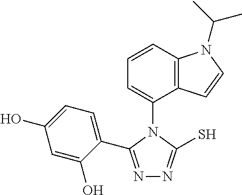 [SMILES: CC(C)n1ccc2c(-n3c(S)nnc3-c3ccc(O)cc3O)cccc21] | 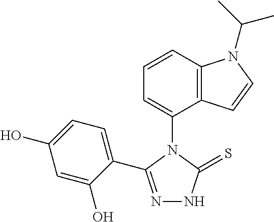 [SMILES: CC(C)n1ccc2c(-n3c(-c4ccc(O)cc4O)n[nH]c3=S)cccc21] | 3-(2,4-DIHYDROXY- PHENYL)-4-(1-ISOPROPYL- INDOL-4-YL)-5- MERCAPTO-[1,2,4] TRIAZOLE |
| 5 | 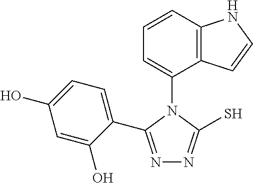 [SMILES: Oc1ccc(-c2nnc(S)n2-c2cccc3[nH]ccc23)c(O)c1] | 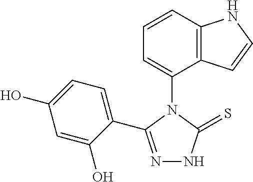 [SMILES: Oc1ccc(-c2n[nH]c(=S)n2-c2cccc3[nH]ccc23)c(O)c1] | 3-(2,4-DIHYDROXY- PHENYL)-4-(INDOL-4-YL)- 5-MERCAPTO-[1,2,4] TRIAZOLE |
| 6 | 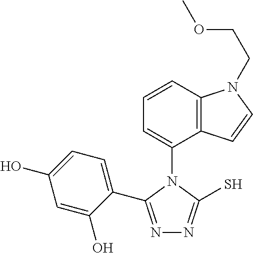 [SMILES: COCCn1ccc2c(-n3c(S)nnc3-c3ccc(O)cc3O)cccc21] | 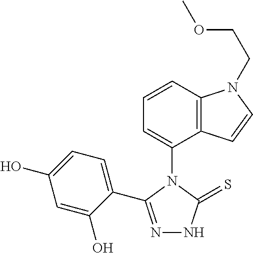 [SMILES: COCCn1ccc2c(-n3c(-c4ccc(O)cc4O)n[nH]c3=S)cccc21] | 3-(2,4-DIHYDROXY- PHENYL)-4-[1-(2- METHOXYETHOXY)- INDOL-4-YL]-5- MERCAPTO-[1,2,4] TRIAZOLE |
| 7 | 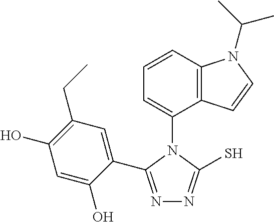 [SMILES: CCc1cc(-c2nnc(S)n2-c2cccc3c2ccn3C(C)C)c(O)cc1O] | 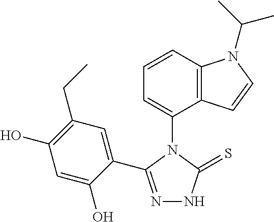 [SMILES: CCc1cc(-c2n[nH]c(=S)n2-c2cccc3c2ccn3C(C)C)c(O)cc1O] | 3-(2,4-DIHYDROXY-5- ETHYL-PHENYL)-4-(1- ISOPROPYL-INDOL-4- YL)-5-MERCAPTO-[1,2,4] TRIAZOLE |
| 8 | 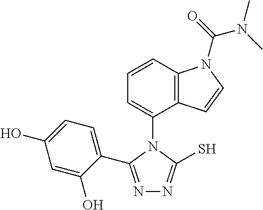 [SMILES: CN(C)C(=O)n1ccc2c(-n3c(S)nnc3-c3ccc(O)cc3O)cccc21] | 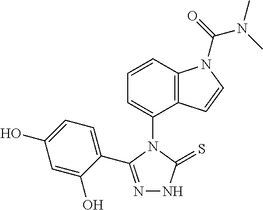 [SMILES: CN(C)C(=O)n1ccc2c(-n3c(-c4ccc(O)cc4O)n[nH]c3=S)cccc21] | 3-(2,4-DIHYDROXY-5- ETHYL-PHENYL)-4-[1- (DIMETHYL-CARBAMOYL)- INDOL-4-YL]-5- MERCAPTO-[1,2,4] TRIAZOLE |
| 9 | 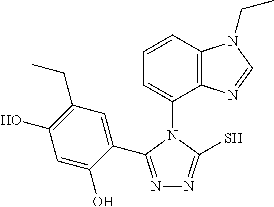 [SMILES: CCc1cc(-c2nnc(S)n2-c2cccc3c2ncn3CC)c(O)cc1O] | 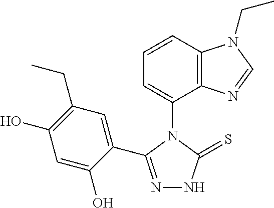 [SMILES: CCc1cc(-c2n[nH]c(=S)n2-c2cccc3c2ncn3CC)c(O)cc1O] | 3-(2,4-DIHYDROXY-5- ETHYL-PHENYL)-4-(1- ETHYL-BENZOIMIDAZOL- 4-YL)-5-MERCAPTO- [1,2,4] TRIAZOLE |
| 10 | 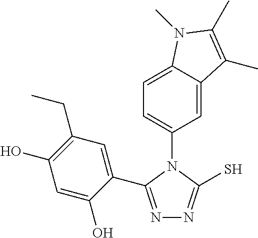 [SMILES: CCc1cc(-c2nnc(S)n2-c2ccc3c(c2)c(C)c(C)n3C)c(O)cc1O] | 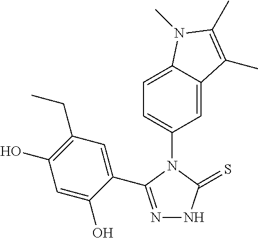 [SMILES: CCc1cc(-c2n[nH]c(=S)n2-c2ccc3c(c2)c(C)c(C)n3C)c(O)cc1O] | 3-(2,4-DIHYDROXY-5- ETHYL-PHENYL)-4-(1,2,3- TRIMETHYL-INDOL- 5-YL)-5-MERCAPTO-[1,2,4] TRIAZOLE |
| 11 | 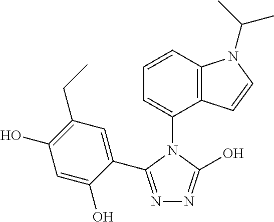 [SMILES: CCc1cc(-c2nnc(O)n2-c2cccc3c2ccn3C(C)C)c(O)cc1O] | 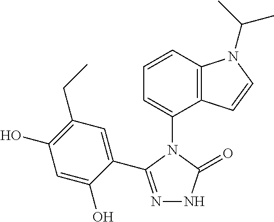 [SMILES: CCc1cc(-c2n[nH]c(=O)n2-c2cccc3c2ccn3C(C)C)c(O)cc1O] | 3-(2,4-DIHYDROXY-5- ETHYL-PHENYL)-4-(1- ISOPROPYL-INDOL-3- YL)-5-HYDROXY-[1,2,4] TRIAZOLE |
| 12 | 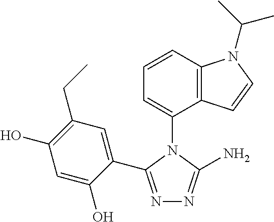 [SMILES: CCc1cc(-c2nnc(N)n2-c2cccc3c2ccn3C(C)C)c(O)cc1O] | 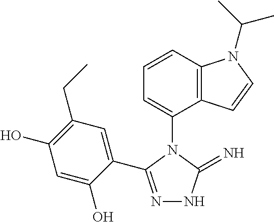 [SMILES: CCc1cc(-c2n[nH]c(=N)n2-c2cccc3c2ccn3C(C)C)c(O)cc1O] | 3-(2,4-DIHYDROXY-5- ETHYL-PHENYL)-4-(1- ISOPROPYL-INDOL-4-YL)- 5-AMINO-[1,2,4] TRIAZOLE |
| 15 | 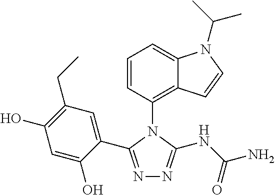 [SMILES: CCc1cc(-c2nnc(NC(N)=O)n2-c2cccc3c2ccn3C(C)C)c(O)cc1O] | | 3-(2,4-DIHYDROXY-5- ETHYL-PHENYL)-4- (1-ISOPROPYL-INDOL- 4-YL)-5-UREIDO-[1,2,4] TRIAZOLE |
| 16 | 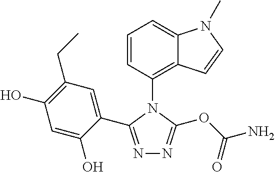 [SMILES: CCc1cc(-c2nnc(OC(N)=O)n2-c2cccc3c2ccn3C)c(O)cc1O] | | 3-(2,4-DIHYDROXY-5- ETHYL-PHENYL)-4- (1-METHYL-INDOL-4- YL)-5-CARBAMOYLOXY- [1,2,4] TRIAZOLE |
| 17 | 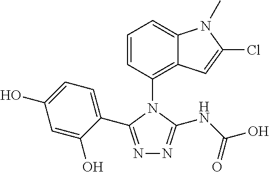 [SMILES: Cn1c(Cl)cc2c(-n3c(NC(=O)O)nnc3-c3ccc(O)cc3O)cccc21] | | 3-(2,4-DIHYDROXY- PHENYL)-4-(1-METHYL- 2-CHLORO-INDOL-4-YL)-5- CARBAMOYLOXY-[1,2,4] TRIAZOLE |
| 18 | 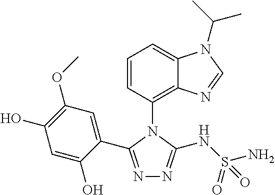 [SMILES: COc1cc(-c2nnc(NS(N)(=O)=O)n2-c2cccc3c2ncn3C(C)C)c(O)cc1O] | | 3-(2,4-DIHYDROXY-5- METHOXY-PHENYL)-4-(1- ISOPROPYL- BENZOIMIDAZOL-4-YL)-5- (SULFAMOYLAMINO)- [1,2,4] TRIAZOLE |
| 20 | 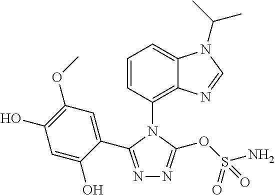 [SMILES: COc1cc(-c2nnc(OS(N)(=O)=O)n2-c2cccc3c2ncn3C(C)C)c(O)cc1O] | | 3-(2,4-DIHYDROXY-5- METHOXY-PHENYL)-4-(1- ISOPROPYL- BENZOIMIDAZOL-4-YL)-5- (SULFAMOYLOXY)- [1,2,4] TRIAZOLE |
| 21 | 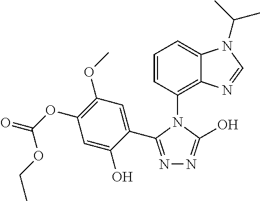 [SMILES: CCOC(=O)Oc1cc(O)c(-c2nnc(O)n2-c2cccc3c2ncn3C(C)C)cc1OC] | 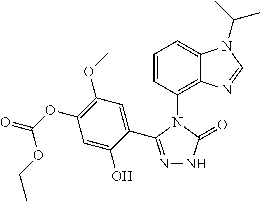 [SMILES: CCOC(=O)Oc1cc(O)c(-c2n[nH]c(=O)n2-c2cccc3c2ncn3C(C)C)cc1OC] | 3-(2-HYDROXY-4- ETHOXYCARBONYOXY-5- METHOXY-PHENYL)-4-(1- ISOPROPYL- BENZOIMIDAZOL-4-YL)-5- HYDROXY-[1,2,4] TRIAZOLE |
| 22 | 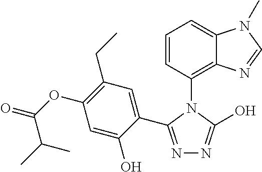 [SMILES: CCc1cc(-c2nnc(O)n2-c2cccc3c2ncn3C)c(O)cc1OC(=O)C(C)C] | 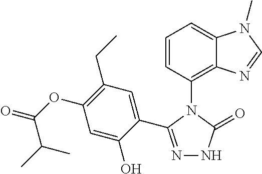 [SMILES: CCc1cc(-c2n[nH]c(=O)n2-c2cccc3c2ncn3C)c(O)cc1OC(=O)C(C)C] | 3-[2-HYDROXY-4- ISOBUTYRYLOXY-5-ETHYL- PHENYL]-4-(1-METHYL- BENZO-IMIDAZOL-4-YL)-5- HYDROXY- [1,2,4] TRIAZOLE |
| 23 | 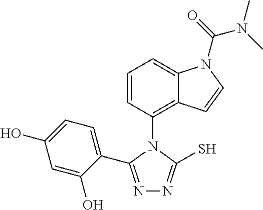 [SMILES: CN(C)C(=O)n1ccc2c(-n3c(S)nnc3-c3ccc(O)cc3O)cccc21] | 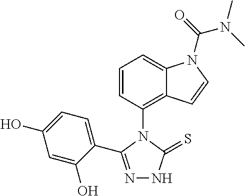 [SMILES: CN(C)C(=O)n1ccc2c(-n3c(-c4ccc(O)cc4O)n[nH]c3=S)cccc21] | 3-(2,4-DIHYDROXY- PHENYL)-4-(1-DIMETHYL- CARBAMOYL-INDOL-4- YL)-5-MERCAPTO- [1,2,4] TRIAZOLE |
| 24 | 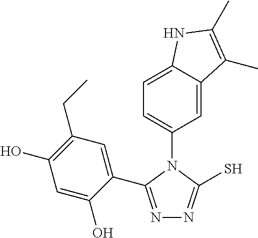 [SMILES: CCc1cc(-c2nnc(S)n2-c2ccc3[nH]c(C)c(C)c3c2)c(O)cc1O] | 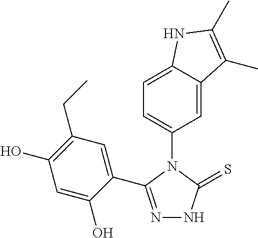 [SMILES: CCc1cc(-c2n[nH]c(=S)n2-c2ccc3[nH]c(C)c(C)c3c2)c(O)cc1O] | 3-(2,4-DIHYDROXY-5- ETHYL-PHENYL)-4-(2,3- DIMETHYL-INDOL-5- YL)-5-MERCAPTO- [1,2,4] TRIAZOLE |
| 25 | 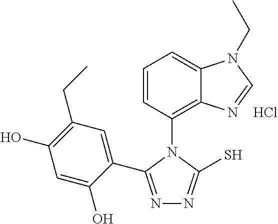 [SMILES: CCc1cc(-c2nnc(S)n2-c2cccc3c2ncn3CC)c(O)cc1O ; Cl] | 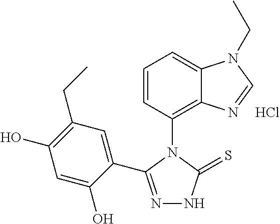 [SMILES: CCc1cc(-c2n[nH]c(=S)n2-c2cccc3c2ncn3CC)c(O)cc1O ; Cl] | 3-(2,4-DIHYDROXY-5- ETHYL-PHENYL)-4-(1- ETHYL-1H- BENZOIMIDAZOL-4- YL)-5-MERCAPTO- [1,2,4] TRIAZOLE, HCL SALT |
| 26 | 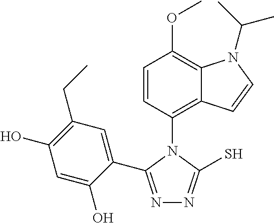 [SMILES: CCc1cc(-c2nnc(S)n2-c2ccc(OC)c3c2ccn3C(C)C)c(O)cc1O] | 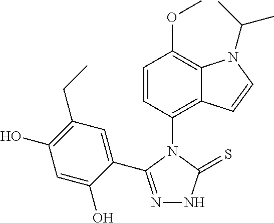 [SMILES: CCc1cc(-c2n[nH]c(=S)n2-c2ccc(OC)c3c2ccn3C(C)C)c(O)cc1O] | 3-(2,4-DIHYDROXY-5- ETHYL-PHENYL)-4-(1- ISOPROPYL-7- METHOXY-INDOL-4- YL)-5-MERCAPTO-[1,2,4] TRIAZOLE |
| 27 | 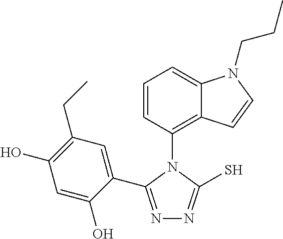 [SMILES: CCCn1ccc2c(-n3c(S)nnc3-c3cc(CC)c(O)cc3O)cccc21] | 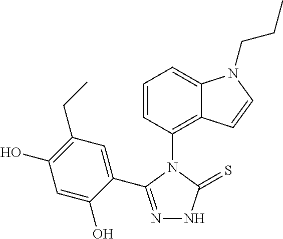 [SMILES: CCCn1ccc2c(-n3c(-c4cc(CC)c(O)cc4O)n[nH]c3=S)cccc21] | 3-(2,4-DIHYDROXY-5- ETHYL-PHENYL)-4-(1- PROPYL-INDOL-4-YL)- 5-MERCAPTO-[1,2,4] TRIAZOLE |
| 28 | 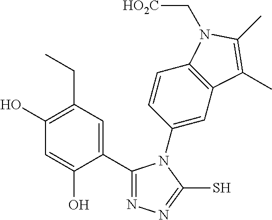 [SMILES: CCc1cc(-c2nnc(S)n2-c2ccc3c(c2)c(C)c(C)n3CC(=O)O)c(O)cc1O] | 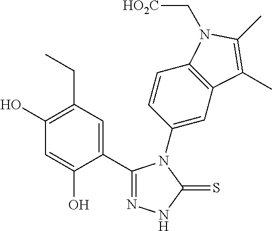 [SMILES: CCc1cc(-c2n[nH]c(=S)n2-c2ccc3c(c2)c(C)c(C)n3CC(=O)O)c(O)cc1O] | 3-(2,4-DIHYDROXY-5- ETHYL-PHENYL)-4-(1- ACETYL-2,3-DIMETHYL- INDOL-5-YL)-5- MERCAPTO-[1,2,4] TRIAZOLE |
| 29 | 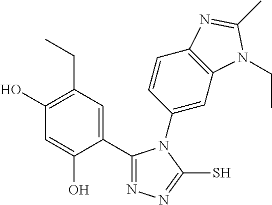 [SMILES: CCc1cc(-c2nnc(S)n2-c2ccc3nc(C)n(CC)c3c2)c(O)cc1O] | 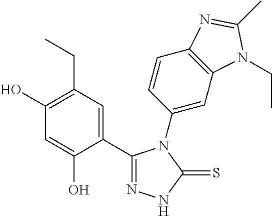 [SMILES: CCc1cc(-c2n[nH]c(=S)n2-c2ccc3nc(C)n(CC)c3c2)c(O)cc1O] | 3-(2,4-DIHYDROXY-5- ETHYL-PHENYL)-4-(2- METHYL-3-ETHYL- BENZIMIDAZOL-5-YL)-5- MERCAPTO-[1,2,4] TRIAZOLE |
| 30 | 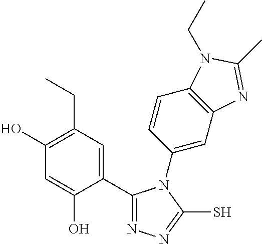 [SMILES: CCc1cc(-c2nnc(S)n2-c2ccc3c(c2)nc(C)n3CC)c(O)cc1O] | 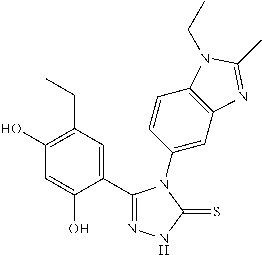 [SMILES: CCc1cc(-c2n[nH]c(=S)n2-c2ccc3c(c2)nc(C)n3CC)c(O)cc1O] | 3-(2,4-DIHYDROXY-5- ETHYL-PHENYL)-4- (1-ETHYL-2-METHYL- BENZIMIDAZOL-5-YL)-5- MERCAPTO-[1,2,4] TRIAZOLE |
| 31 | 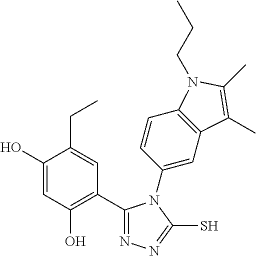 [SMILES: CCCn1c(C)c(C)c2cc(-n3c(S)nnc3-c3cc(CC)c(O)cc3O)ccc21] | 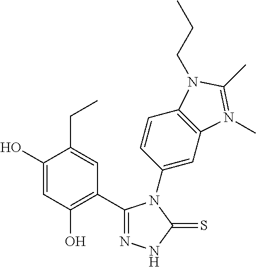 | 3-(2,4-DIHYDROXY-5- ETHYL-PHENYL)-4-(1- PROPYL-2,3-DIMETHYL- INDOL-5-YL)-5-MERCAPTO- [1,2,4] TRIAZOLE |
| 34 | 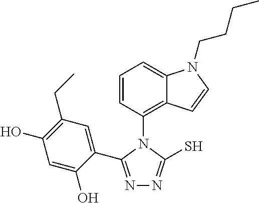 [SMILES: CCCCn1ccc2c(-n3c(S)nnc3-c3cc(CC)c(O)cc3O)cccc21] | 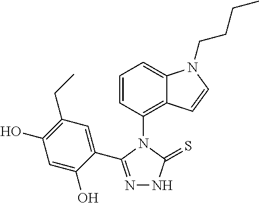 [SMILES: CCCCn1ccc2c(-n3c(-c4cc(CC)c(O)cc4O)n[nH]c3=S)cccc21] | 3-(2,4-DIHYDROXY-5- ETHYL-PHENYL)-4-(1- N-BUTYL-INDOL-4-YL)- 5-MERCAPTO- [1,2,4] TRIAZOLE |
| 35 | 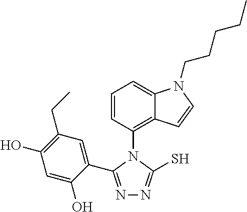 [SMILES: CCCCCn1ccc2c(-n3c(S)nnc3-c3cc(CC)c(O)cc3O)cccc21] | 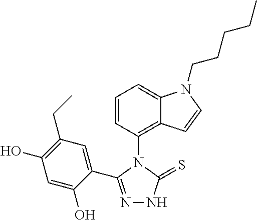 [SMILES: CCCCCn1ccc2c(-n3c(-c4cc(CC)c(O)cc4O)n[nH]c3=S)cccc21] | 3-(2,4-DIHYDROXY-5- ETHYL-PHENYL)-4-(1- N-PENTYL-INDOL-4- YL)-5-MERCAPTO- [1,2,4] TRIAZOLE |
| 36 | 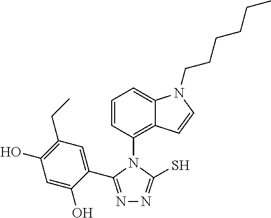 [SMILES: CCCCCCn1ccc2c(-n3c(S)nnc3-c3cc(CC)c(O)cc3O)cccc21] | 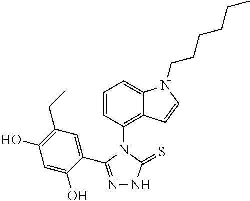 [SMILES: CCCCCCn1ccc2c(-n3c(-c4cc(CC)c(O)cc4O)n[nH]c3=S)cccc21] | 3-(2,4-DIHYDROXY-5- ETHYL-PHENYL)-4-(1- N-HEXYL-INDOL-4-YL)- 5-MERCAPTO- [1,2,4] TRIAZOLE |
| 37 | 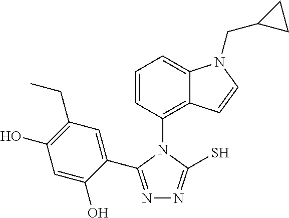 [SMILES: CCc1cc(-c2nnc(S)n2-c2cccc3c2ccn3CC2CC2)c(O)cc1O] | 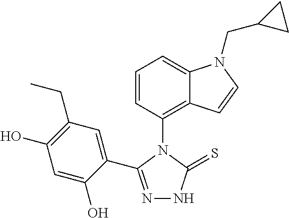 [SMILES: CCc1cc(-c2n[nH]c(=S)n2-c2cccc3c2ccn3CC2CC2)c(O)cc1O] | 3-(2,4-DIHYDROXY-5- CYCLOPROPYL-PHENYL)- 4-(1-(1-METHYL- CYCLOPROPYL)-INDOL- 4-YL)-5-MERCAPTO- [1,2,4] TRIAZOLE |
| 38 | 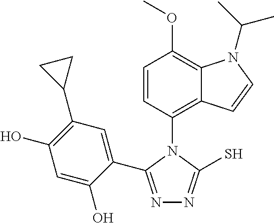 [SMILES: COc1ccc(-n2c(S)nnc2-c2cc(C3CC3)c(O)cc2O)c2ccn(C(C)C)c12] | 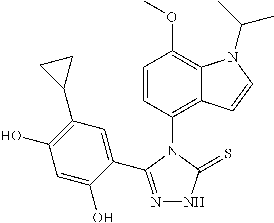 [SMILES: COc1ccc(-n2c(-c3cc(C4CC4)c(O)cc3O)n[nH]c2=S)c2ccn(C(C)C)c12] | 3-(2,4-DIHYDROXY-5- CYCLOPROPYL-PHENYL)-4- (1-ISOPROPYL-7-METHOXY- INDOL-4-YL)-5-MERCAPTO- [1,2,4] TRIAZOLE |
| 39 | 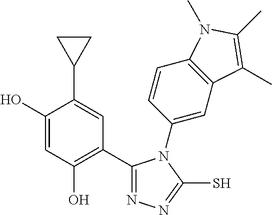 [SMILES: Cc1c(C)n(C)c2ccc(-n3c(S)nnc3-c3cc(C4CC4)c(O)cc3O)cc12] | 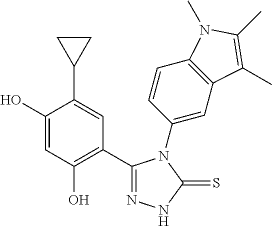 [SMILES: Cc1c(C)n(C)c2ccc(-n3c(-c4cc(C5CC5)c(O)cc4O)n[nH]c3=S)cc12] | 3-(2,4-DIHYDROXY-5- CYCLOPROPYL-PHENYL)- 4-(1,2,3-TRIMETHYL- INDOL-5-YL)-5-MERCAPTO- [1,2,4] TRIAZOLE |
| 40 | 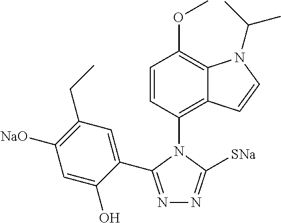 | | 3-(2,4-DIHYDROXY-5- ETHYL-PHENYL)-4-(1- ISOPROPYL-7-METHOXY- INDOL-4-YL)-5- MERCAPTO-[1,2,4] TRIAZOLE DISODIUM SALT |
| 41 | 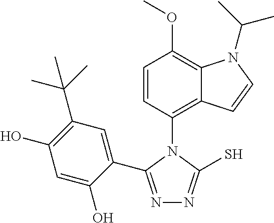 [SMILES: COc1ccc(-n2c(S)nnc2-c2cc(C(C)(C)C)c(O)cc2O)c2ccn(C(C)C)c12] | 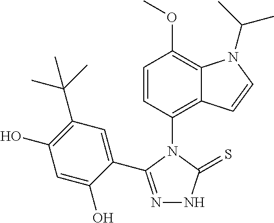 [SMILES: COc1ccc(-n2c(-c3cc(C(C)(C)C)c(O)cc3O)n[nH]c2=S)c2ccn(C(C)C)c12] | 3-(2,4-DIHYDROXY-5-TERT- BUTYL-PHENYL)-4-(1- ISOPROPYL-7-METHOXY- INDOL-4-YL)-5-MERCAPTO- [1,2,4] TRIAZOLE |
| 42 | 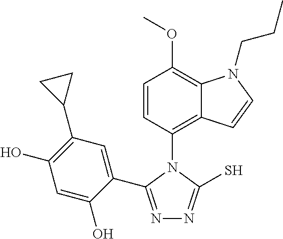 [SMILES: CCCn1ccc2c(-n3c(S)nnc3-c3cc(C4CC4)c(O)cc3O)ccc(OC)c21] | 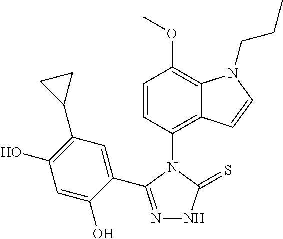 [SMILES: CCCn1ccc2c(-n3c(-c4cc(C5CC5)c(O)cc4O)n[nH]c3=S)ccc(OC)c21] | 3-(2,4-DIHYDROXY-5- CYCLOPROPYL-PHENYL)- 4-(1-PROPYL-7- METHOXY-INDOL- 4-YL)-5-MERCAPTO-[1,2,4] TRIAZOLE |
| 43 | 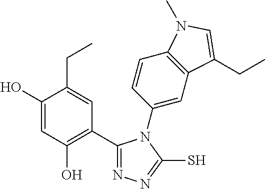 [SMILES: CCc1cc(-c2nnc(S)n2-c2ccc3c(c2)c(CC)cn3C)c(O)cc1O] | 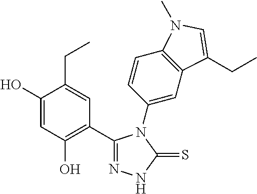 [SMILES: CCc1cc(-c2n[nH]c(=S)n2-c2ccc3c(c2)c(CC)cn3C)c(O)cc1O] | 3-(2,4-DIHYDROXY-5- ETHYL-PHENYL)-4-(1 METHYL-3-ETHYL- INDOL-5-YL)-5- MERCAPTO-[1,2,4] TRIAZOLE |
| 44 | 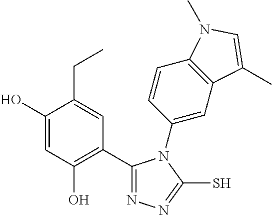 [SMILES: CCc1cc(-c2nnc(S)n2-c2ccc3c(c2)c(C)cn3C)c(O)cc1O] | 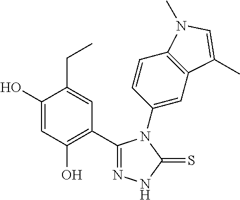 [SMILES: CCc1cc(-c2n[nH]c(=S)n2-c2ccc3c(c2)c(C)cn3C)c(O)cc1O] | 3-(2,4-DIHYDROXY-5- ETHYL-PHENYL)-4-(1,3- DIMETHYL-INDOL-5- YL)-5-MERCAPTO- [1,2,4] TRIAZOLE |
| 45 | 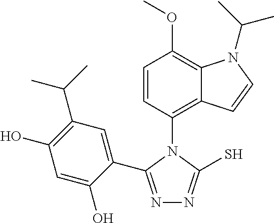 [SMILES: COc1ccc(-n2c(S)nnc2-c2cc(C(C)C)c(O)cc2O)c2ccn(C(C)C)c12] | 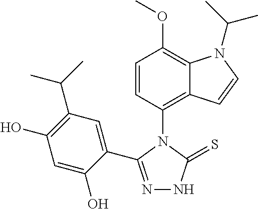 [SMILES: COc1ccc(-n2c(-c3cc(C(C)C)c(O)cc3O)n[nH]c2=S)c2ccn(C(C)C)c12] | 3-(2,4-DIHYDROXY-5- ISOPROPYL-PHENYL)-4-(1- ISOPROPYL-7-METHOXY- INDOL-4-YL)-5-MERCAPTO- [1,2,4] TRIAZOLE |
| 46 | 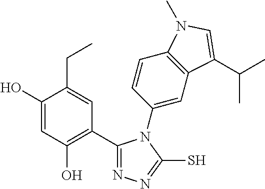 [SMILES: CCc1cc(-c2nnc(S)n2-c2ccc3c(c2)c(C(C)C)cn3C)c(O)cc1O] | 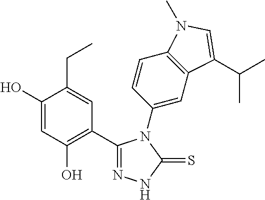 [SMILES: CCc1cc(-c2n[nH]c(=S)n2-c2ccc3c(c2)c(C(C)C)cn3C)c(O)cc1O] | 3-(2,4-DIHYDROXY-5- ETHYL-PHENYL)-4-(1- METHYL-3-ISOPROPYL- INDOL-5-YL)-5-MERCAPTO- [1,2,4] TRIAZOLE |
| 48 | 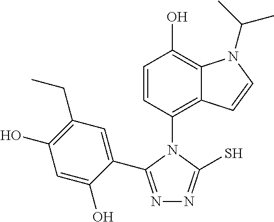 [SMILES: CCc1cc(-c2nnc(S)n2-c2ccc(O)c3c2ccn3C(C)C)c(O)cc1O] | 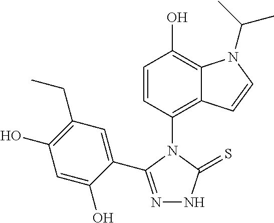 [SMILES: CCc1cc(-c2n[nH]c(=S)n2-c2ccc(O)c3c2ccn3C(C)C)c(O)cc1O] | 3-(2,4-DIHYDROXY-5- ETHYL-PHENYL)-4-(1- ISOPROPYL-7-HYDROXY- INDOL-4-YL)-5-MERCAPTO- [1,2,4] TRIAZOLE |
| 49 | 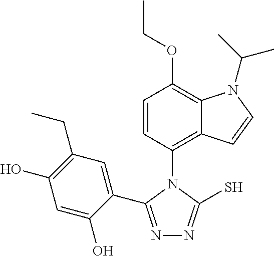 [SMILES: CCOc1ccc(-n2c(S)nnc2-c2cc(CC)c(O)cc2O)c2ccn(C(C)C)c12] | 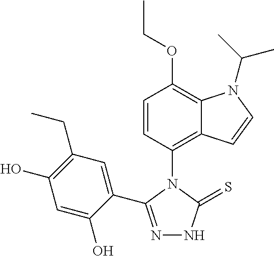 [SMILES: CCOc1ccc(-n2c(-c3cc(CC)c(O)cc3O)n[nH]c2=S)c2ccn(C(C)C)c12] | 3-(2,4-DIHYDROXY-5- ETHYL-PHENYL)-4-(1- ISOPROPYL-7-ETHOXY- INDOL-4-YL)-5-MERCAPTO- [1,2,4] TRIAZOLE |
| 50 | 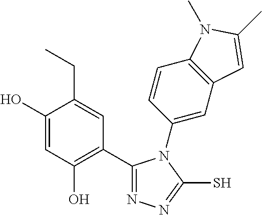 [SMILES: CCc1cc(-c2nnc(S)n2-c2ccc3c(c2)cc(C)n3C)c(O)cc1O] | 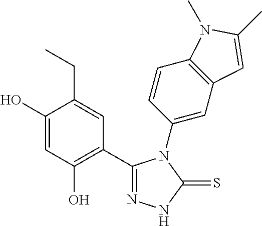 [SMILES: CCc1cc(-c2n[nH]c(=S)n2-c2ccc3c(c2)cc(C)n3C)c(O)cc1O] | 3-(2,4-DIHYDROXY-5- ETHYL-PHENYL)-4-(1,2- DIMETHYL-INDOL-5-YL)-5- MERCAPTO-[1,2,4] TRIAZOLE |
| 51 | 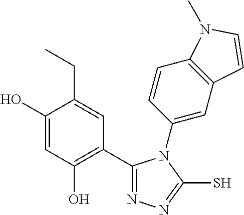 [SMILES: CCc1cc(-c2nnc(S)n2-c2ccc3c(ccn3C)c2)c(O)cc1O] | 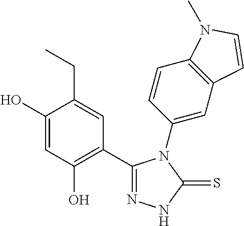 [SMILES: CCc1cc(-c2n[nH]c(=S)n2-c2ccc3c(ccn3C)c2)c(O)cc1O] | 3-(2,4-DIHYDROXY-5- ETHYL-PHENYL)-4-(N- METHYL-INDOL-5-YL)- 5-MERCAPTO-[1,2,4] TRIAZOLE |
| 55 | 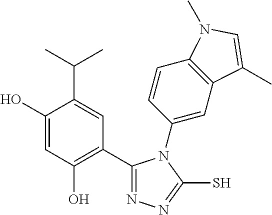 [SMILES: Cc1cn(C)c2ccc(-n3c(S)nnc3-c3cc(C(C)C)c(O)cc3O)cc12] | 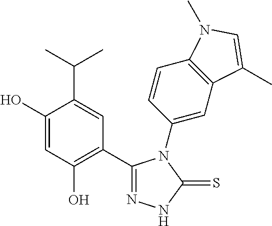 [SMILES: Cc1cn(C)c2ccc(-n3c(-c4cc(C(C)C)c(O)cc4O)n[nH]c3=S)cc12] | 3-(2,4-DIHYDROXY-5- ISOPROPYL-PHENYL)-4-(1,3- DIMETHYL-INDOL-5-YL)-5- MERCAPTO-[1,2,4] TRIAZOLE |
| 56 | 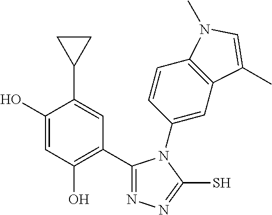 [SMILES: Cc1cn(C)c2ccc(-n3c(S)nnc3-c3cc(C4CC4)c(O)cc3O)cc12] | 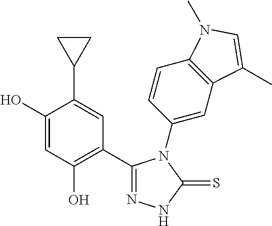 [SMILES: Cc1cn(C)c2ccc(-n3c(-c4cc(C5CC5)c(O)cc4O)n[nH]c3=S)cc12] | 3-(2,4-DIHYDROXY-5- CYCLOPROPYL-PHENYL)- 4-(1,3-DIMETHYL-INDOL- 5-YL)-5-MERCAPTO- [1,2,4] TRIAZOLE |
| 57 | 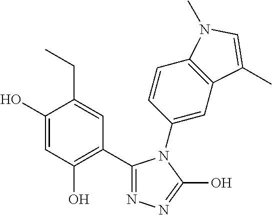 [SMILES: CCc1cc(-c2nnc(O)n2-c2ccc3c(c2)c(C)cn3C)c(O)cc1O] | 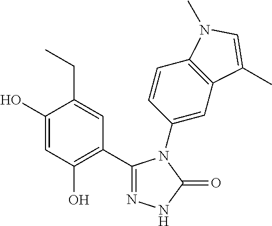 [SMILES: CCc1cc(-c2n[nH]c(=O)n2-c2ccc3c(c2)c(C)cn3C)c(O)cc1O] | 3-(2,4-DIHYDROXY-5- ETHYL-PHENYL)-4-(1,3- DIMETHYL-INDOL-5-YL)- 5-HYDROXY-[1,2,4] TRIAZOLE |
| 58 | 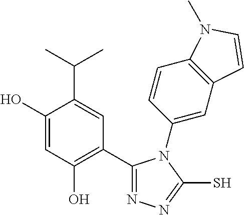 [SMILES: CC(C)c1cc(-c2nnc(S)n2-c2ccc3c(ccn3C)c2)c(O)cc1O] | 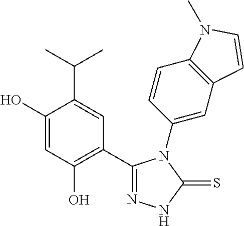 [SMILES: CC(C)c1cc(-c2n[nH]c(=S)n2-c2ccc3c(ccn3C)c2)c(O)cc1O] | 3-(2,4-DIHYDROXY-5- ISOPROPYL-PHENYL)-4-(N- METHYL-INDOL-5-YL)-5- MERCAPTO-[1,2,4] TRIAZOLE |
| 59 | 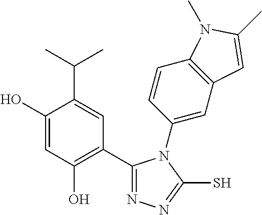 [SMILES: Cc1cc2cc(-n3c(S)nnc3-c3cc(C(C)C)c(O)cc3O)ccc2n1C] | 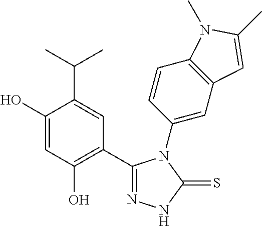 [SMILES: Cc1cc2cc(-n3c(-c4cc(C(C)C)c(O)cc4O)n[nH]c3=S)ccc2n1C] | 3-(2,4-DIHYDROXY-5- ISOPROPYL-PHENYL)-4-(1,2- DIMETHYL-INDOL-5-YL)-5- MERCAPTO-[1,2,4] TRIAZOLE |
| 60 | 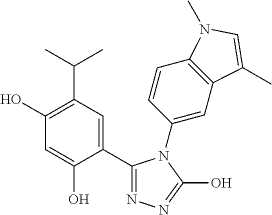 [SMILES: Cc1cn(C)c2ccc(-n3c(O)nnc3-c3cc(C(C)C)c(O)cc3O)cc12] | 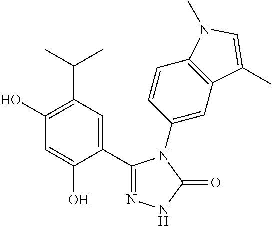 [SMILES: Cc1cn(C)c2ccc(-n3c(-c4cc(C(C)C)c(O)cc4O)n[nH]c3=O)cc12] | 3-(2,4-DIHYDROXY-5- ISOPROPYL-PHENYL)-4- (1,3-DIMETHYL-INDOL-5- YL)-5-HYDROXY-[1,2,4] TRIAZOLE |
| 62 | 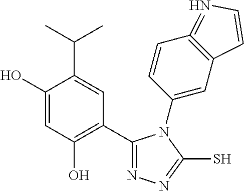 | 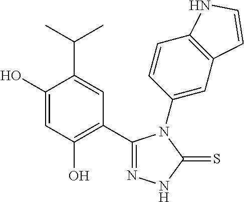 [SMILES: CC(C)c1cc(-c2n[nH]c(=S)n2-c2ccc3[nH]ccc3c2)c(O)cc1O] | 3-(2,4-DIHYDROXY-5- ISOPROPYL-PHENYL)-4- (1H-INDOL-5-YL)-5- MERCAPTO-[1,2,4] TRIAZOLE |
| 63 | 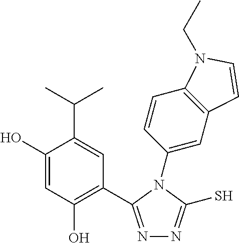 [SMILES: CCn1ccc2cc(-n3c(S)nnc3-c3cc(C(C)C)c(O)cc3O)ccc21] | 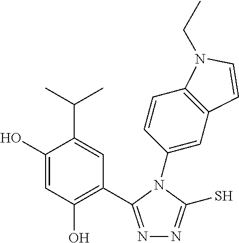 [SMILES: CCn1ccc2cc(-n3c(S)nnc3-c3cc(C(C)C)c(O)cc3O)ccc21] | 3-(2,4-DIHYDROXY-5- ISOPROPYL-PHENYL)-4-(1- ETHYL-INDOL-5-YL)-5- MERCAPTO-[1,2,4] TRIAZOLE |
| 64 | 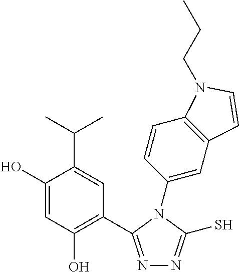 [SMILES: CCCn1ccc2cc(-n3c(S)nnc3-c3cc(C(C)C)c(O)cc3O)ccc21] | 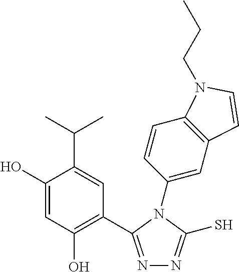 [SMILES: CCCn1ccc2cc(-n3c(S)nnc3-c3cc(C(C)C)c(O)cc3O)ccc21] | 3-(2,4-DIHYDROXY-5- ISOPROPYL-PHENYL)-4- (1-PROPYL-INDOL-5-YL)- 5-MERCAPTO-[1,2,4] TRIAZOLE |
| 65 | 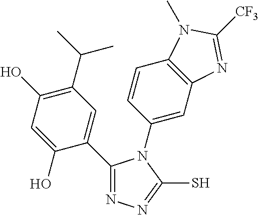 [SMILES: CC(C)c1cc(-c2nnc(S)n2-c2ccc3c(c2)nc(C(F)(F)F)n3C)c(O)cc1O] | 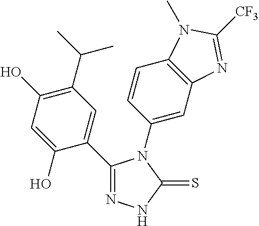 [SMILES: CC(C)c1cc(-c2n[nH]c(=S)n2-c2ccc3c(c2)nc(C(F)(F)F)n3C)c(O)cc1O] | 3-(2,4-DIHYDROXY-5- ISOPROPYL-PHENYL)-4-(1- METHYL-2-TRIFLUORO- METHYL-BENZIMIDAZOL- 5-YL)-5-MERCAPTO-[1,2,4] TRIAZOLE |
| 66 | 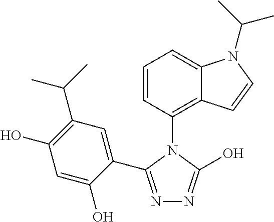 [SMILES: CC(C)c1cc(-c2nnc(O)n2-c2cccc3c2ccn3C(C)C)c(O)cc1O] | 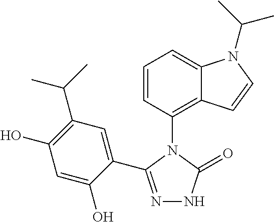 [SMILES: CC(C)c1cc(-c2n[nH]c(=O)n2-c2cccc3c2ccn3C(C)C)c(O)cc1O] | 3-(2,4-DIHYDROXY-5- ISOPROPYL-PHENYL)-4-(1- ISOPROPYL-INDOL-4-YL)- 5-HYDROXY-[1,2,4] TRIAZOLE |

TABLE 2
Compounds according to Formula (Ia)

| NO. | STRUCTURE | TAUTOMERIC STRUCTURE | NAME |
|---|---|---|---|
| 1A | 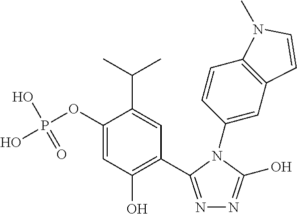 [SMILES: CC(C)c1cc(-c2nnc(O)n2-c2ccc3c(ccn3C)c2)c(O)cc1OP(=O)(O)O] | 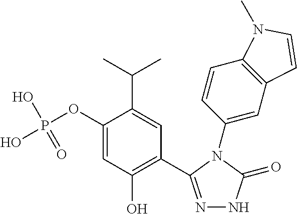 [SMILES: CC(C)c1cc(-c2n[nH]c(=O)n2-c2ccc3c(ccn3C)c2)c(O)cc1OP(=O)(O)O] | 5-HYDROXY-4-(5- HYDROXY-4-(1-METHYL- 1H-INDOL-5-YL)-4H- 1,2,4-TRIAZOL-3-YL)-2- ISOPROPYLPHENYL DIHYDROGEN PHOSPHATE |
| 2A | 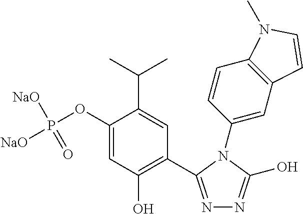 | 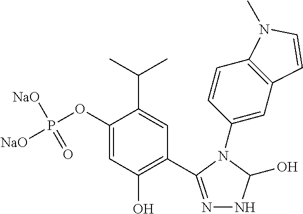 | SODIUM 5-HYDROXY-4- (5-HYDROXY-4-(1- METHYL-1H-INDOL-5- YL)-4H-1,2,4-TRIAZOL-3- YL)-2-ISOPROPYLPHENYL PHOSPHATE |
| 3A | 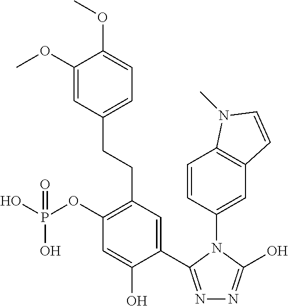 [SMILES: COc1ccc(CCc2cc(-c3nnc(O)n3-c3ccc4c(ccn4C)c3)c(O)cc2OP(=O)(O)O)cc1OC] | 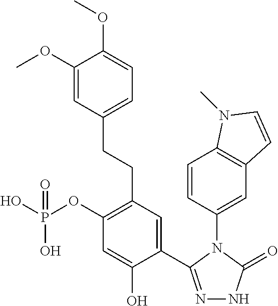 [SMILES: COc1ccc(CCc2cc(-c3n[nH]c(=O)n3-c3ccc4c(ccn4C)c3)c(O)cc2OP(=O)(O)O)cc1OC] | 2-(3,4- DIMETHOXYPHENETHYL)- 5-HYDROXY-4-(5- HYDROXY-4-(1-METHYL- 1H-INDOL-5-YL)-4H- 1,2,4-TRIAZOL-3- YL)PHENYL DIHYDROGEN PHOSPHATE |
| 4A | 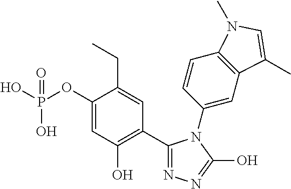 [SMILES: CCc1cc(-c2nnc(O)n2-c2ccc3c(c2)c(C)cn3C)c(O)cc1OP(=O)(O)O] | 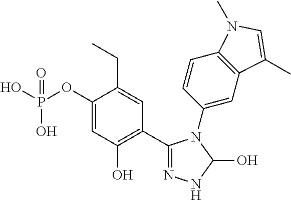 [SMILES: CCc1cc(C2=NNC(O)N2c2ccc3c(c2)c(C)cn3C)c(O)cc1OP(=O)(O)O] | 4-(4-(1,3-DIMETHYL-1H- INDOL-5-YL)-5- HYDROXY-4H-1,2,4- TRIAZOL-3-YL)-2-ETHYL- 5-HYDROXYPHENYL DIHYDROGEN PHOSPHATE |

The Hsp90 inhibitory compounds used in the disclosed combination methods can be prepared according to the procedures disclosed in U.S. Patent Publication No. 2006/0167070, and WO2009/023211.

These triazolone compounds typically can form a tautomeric structure as shown below and as exemplified by the tautomeric structures shown in Tables 1 and 2:

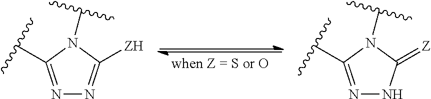

The present invention provides pharmaceutical combinations for the treatment, prophylaxis, and amelioration of proliferative disorders, such as cancer. In a specific embodiment, the combination comprises one or more Hsp90 inhibitors according to formulae (I) or (Ia), or a compound in Tables 1 or 2, or a tautomer or a pharmaceutically acceptable salt thereof in addition to a topoisomerase II inhibitor. Suitable pharmaceutical formulations are described in International Application No. PCT/US201137283, filed May 20, 2011 entitled FORMULATION AND DOSING OF HSP90 INHIBITORY COMPOUNDS, the entire teachings of which are incorporated herein by reference.

In one embodiment, the combination includes a pharmaceutical composition or a single unit dosage form containing both an Hsp90 inhibitor and a topoisomerase II inhibitor. Pharmaceutical combinations and dosage forms described herein comprise the two active ingredients in relative amounts and formulated in such a way that a given pharmaceutical combination or dosage form can be used to treat proliferative disorders, such as cancer. Preferred pharmaceutical combinations and dosage forms comprise a compound of formulae (I) or (Ia), or a compound in Tables 1 or 2, or a tautomer or pharmaceutically acceptable salt thereof, in combination with a topoisomerase II inhibitor. In other embodiments, the Hsp90 inhibitor and the topoisomerase II inhibitor may be in individual or separate pharmaceutical compositions, depending on the dosing schedules, preferred routes of administration, and available formulations of the two inhibitors. Optionally, these embodiments can also contain one or more additional therapeutic agents.

The pharmaceutical combinations described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the combination is formulated in accordance with routine procedures as a pharmaceutical composition adapted for intravenous, subcutaneous, intramuscular, oral, intranasal or topical administration to human beings. In one embodiment, the combination is formulated in accordance with routine procedures for subcutaneous administration to human beings.

In a specific embodiment, the combination therapies described herein comprise one or more compounds and at least one other therapy which has the same mechanism of action as the compounds. In another specific embodiment, the combination therapies described herein comprise one or more compounds described herein and at least one other therapy which has a different mechanism of action than the compounds. In certain embodiments, the combination therapies described herein improve the therapeutic effect of one or more triazolone compounds described herein by functioning together with the topoisomerase II inhibitor to have an additive or synergistic effect. In certain embodiments, the combination therapies described herein reduce the side effects associated with the therapies. In certain embodiments, the combination therapies described herein reduce the effective dosage of one or more of the therapies.

In a specific embodiment, the combination comprising one or more triazolone compounds described herein is administered to a subject, preferably a human, to prevent, treat, manage, or ameliorate cancer, or one or more symptom thereof. In accordance with the invention, the pharmaceutical combinations described herein may also comprise one or more other agents being used, have been used, or are known to be useful in the treatment or amelioration of cancer, particularly non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma. The pharmaceutical combinations described herein utilize pharmaceutical compositions and dosage forms which comprise one or more excipients. Suitable excipients are well known to those skilled in the art of pharmacy.

The triazolone compounds described herein can be also formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

The present invention also provides a method of treating a proliferative disorder in a subject, comprising administering to the subject an effective amount of the combination of an Hsp90 inhibitor and a topoisomerase II inhibitor as described herein. In one embodiment, the proliferative disorder is cancer. In one aspect of this embodiment, the cancer is non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

Smooth muscle cell proliferation includes hyperproliferation of cells in the vasculature, for example, intimal smooth muscle cell hyperplasia, restenosis and vascular occlusion, particularly stenosis following biologically- or mechanically-mediated vascular injury, e.g., vascular injury associated with angioplasty. Moreover, intimal smooth muscle cell hyperplasia can include hyperplasia in smooth muscle other than the vasculature, e.g., bile duct blockage, bronchial airways of the lung in patients with asthma, in the kidneys of patients with renal interstitial fibrosis, and the like.

In one embodiment, the disclosed method is believed to be effective in treating a subject with non-solid tumors such as multiple myeloma. In another embodiment, the disclosed method is believed to be effective against T-cell leukemia, e.g., as exemplified by Jurkat and CEM cell lines; B-cell leukemia, e.g., as exemplified by the SB cell line; promyelocytes, e.g., as exemplified by the HL-60 cell line; uterine sarcoma, e.g., as exemplified by the MES-SA cell line; monocytic leukemia, e.g., as exemplified by the THP-1 (acute) cell line; and lymphoma, e.g., as exemplified by the U937 cell line.

Some of the disclosed methods can be also effective at treating subjects whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including gamma-radiation, neutron beam radiotherapy, electron beam radiotherapy, proton therapy, brachytherapy, and systemic radioactive isotopes), endocrine therapy, biologic response modifiers (including interferons, interleukins, and tumor necrosis factor (TNF)), hyperthermia and cryotherapy, agents to attenuate any adverse effects (e.g., antiemetics), and other approved chemotherapeutic drugs.

The therapeutic agents of the combination therapies described herein can be administered sequentially or concurrently. In one embodiment, the administration of the Hsp90 inhibitor and the topoisomerase II inhibitor are done concurrently. In another embodiment, the administration of the Hsp90 inhibitor and the topoisomerase II inhibitor are done separately. In another embodiment, the administration of the Hsp90 inhibitor and the topoisomerase II inhibitor are done sequentially. In one embodiment, the administration of the Hsp90 inhibitor and the topoisomerase II inhibitor are done until the cancer is cured or stabilized or improved.

In one specific embodiment, the present method includes treating, managing, or ameliorating cancer, or one or more symptoms thereof, comprising administering to a subject in need thereof one or more compounds represented by the structural formulae (I) or (Ia) or a compound in Table 1 or Table 2, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of etoposide.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of etoposide.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In yet another embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In one embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with etoposide.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with etoposide.

In one embodiment, the method of treating a subject with cancer, wherein the subject is being or has been treated with a chemotherapeutic agent, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In one embodiment, the method of treating a subject with cancer, wherein the subject is being or has been treated with a chemotherapeutic agent, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In one embodiment, the method of treating a subject with cancer, wherein the subject has proven refractory to other therapies but is no longer on these therapies, includes administering to the subject an effective amount of a triazolone compound represented by the structural formulae (I) or (Ia) or a compound in Table 1 or Table 2, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with etoposide.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with etoposide.

In one embodiment, the method of treating a subject with cancer, wherein the subject has proven refractory to other therapies but is no longer on these therapies, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In one embodiment, the method of treating a subject with cancer, wherein the subject has proven refractory to other therapies but is no longer on these therapies, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin, wherein the cancer is selected from the group consisting of non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, Non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, and AID's related Karposi's sarcoma.

In one further embodiment, the method includes inhibiting the growth of a cancer or tumor cell comprising the steps of: (a) contacting the cell with an effective amount of a compound of formulae (I) or (Ia) or a compound in Table (1) or Table (2), or tautomer or a pharmaceutically acceptable salt thereof; and (b) exposing the cell to an effective amount of a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of etoposide.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of a topoisomerase II inhibitor such as etoposide, HU-331, ICRF-187 (dexrazoxane), ICRF-193, amsacrine, mitindomide, teniposide, doxorubicin, daunorubicin, idarubicin, mitoxantrone, anteniposide or novobiocin.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of etoposide.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, when the triazolone compounds described herein are administered in combination with a topoisomerase II inhibitor, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies are administered within the same patient visit.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating a proliferative disorders, such as cancer, or one or more symptoms thereof, the methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

### EXAMPLES

### Example 1

### Inhibition of Topoisomerase II

The ability of compounds of the formulae (I) or (Ia) to inhibit the activity of topoisomerase II was examined with a kDNA decatenation assay (TopoGEN, Inc. Port Orange, Fla.). Substrate kDNA was mixed with compounds and incubated at 37° C. for 30 min. The reaction was stop by adding ⅕ volume of stop buffer. 20 μl of the reaction was loaded on 1% agarose gel. Image of decatenation of kDNA by compounds was taken by Kodak Image Station 440. Table 1 indicates the ability of compound 1 inhibiting the activity of topoisomerase II.

TABLE 1

| Compound | Topo II assay |
|---|---|
| 1 | + |

Effectiveness at inhibition: + (some inhibition) < ++ < +++ < ++++ (complete inhibition)

### Example 2

### Combination Studies with Compound 1 and Etoposide

### A. Materials and Methods

### Cell Lines

Human K562 chronic myelogenous leukemia cells (American Type Culture Collection) were grown in RPMI medium with 2 mM L-glutamine, antibiotics (100 IU/ml penicillin and 100 μg/ml streptomycin) and 10% fetal bovine serum (Sigma Aldrich). Cells were maintained at 37° C., 5% CO₂ atmosphere and subcultured at 1×10⁶ cells/mL.

### Cell Viability Assays

Cell viability was measured using the Alamar Blue assay (Invitrogen). In brief, cells were plated in 96-well plates in triplicate at 2000 cells per well and incubated at 37° C., 5% CO₂ atmosphere for 24 hr prior to the addition of drug or vehicle (0.3% DMSO) to the culture medium. After 72 hr, 10 μl/well Alamar Blue was added to the wells and incubated for an additional 3 hr at 37° C., 5% CO₂ atmosphere. Fluorescence (560_{Ex}/590_{Em} nM) was measured with a SpectraMax microplate reader (Molecular Devices) and the resulting data were used to calculate cell viability, normalized to vehicle control. FIG. 1A showed the dose response curve for compound 1, and FIG. 1B showed the dose response curve for etoposide as single agents.

B. Combination Studies with Etoposide and Compound 1

The half maximal inhibitory concentration (IC₅₀) for etoposide or Compound 1 was first determined using a 1.5-fold serial dilution series of compound. After 72 hr exposure to drug, cell viability was measured and results were fit to a four parameter logistic model (XLFit, ID Business Solutions) shown in FIG. 1. The IC₅₀ for Compound 1 was calculated at 16 nM, and etoposide at 21 nM.

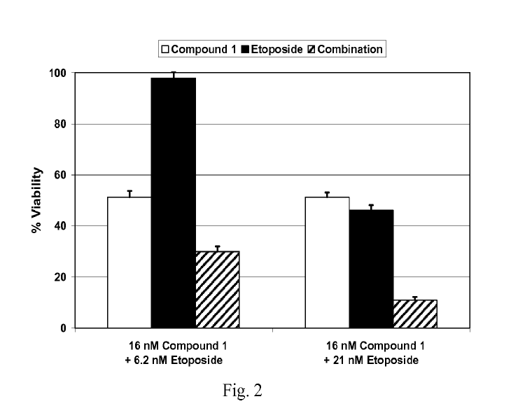

Combinations between etoposide and Compound 1 were then performed concurrently based on the IC₅₀ for each agent. The combined drugs, as well as each drug alone, were incubated with the cells for 3 days and the surviving fraction of cells relative to control was determined using the Alamar Blue assay. Shown in FIG. 2, the combination of etoposide with Compound 1 displayed significantly enhanced cytotoxicity relative to either agent alone. It is thus believed the combination therapy had great potential in the treatment of cancer.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A pharmaceutical combination comprising a topoisomerase II inhibitor and an Hsp90 inhibitor according to the following formulae: 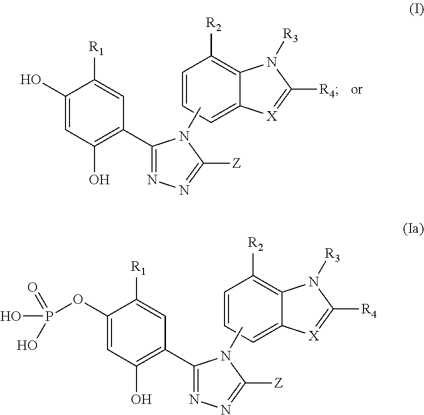
or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

2-3. (canceled)

4. The combination of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

5. The combination of claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

6. The combination according to claim 1, wherein the topoisomerase II inhibitor is selected from the group consisting of etoposide, amsacrine, mitindomide, teniposide, doxorubicin, daunorubicin, idarubicin, mitoxantrone, anteniposide, novobiocin, dexrazoxane, 3-hydroxy-2-[(1R)-6-isopropenyl-3-methyl-cyclohex-2-en-1-yl]-5-pentyl-1,4-benzoquinone, and 4-[2-(3,5-dioxo-1-piperazinyl)-1-methylpropyl]piperazine-2,6-dione.

7. The combination according to claim 6, wherein the topoisomerase II inhibitor is etoposide.

8. The combination according to claim 1, wherein the Hsp90 inhibitor is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof, and the topoisomerase II inhibitor is etoposide.

9. The combination according to claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the topoisomerase II inhibitor is etoposide.

10. The combination according to claim 1, further comprising one or more additional therapeutic agents.

11. The combination of claim 10, wherein the one or more therapeutic agents are selected from the group consisting of vandetanib, trastuzumab, temodar, irinotecan, dexamethasone, cisplatin, epirubicin, ifosfamide, oxaliplatin, mitoxantrone, vorinostat, carboplatin, interferon alpha, rituximab, prednisone, cyclophosphamide, bendamustine, adriamycin, valproate, celecoxib, thalidomide, nelarabine, methotrexate, filgrastim, gemtuzumab ozogamicin, testosterone, clofarabine, cytarabine, everolimus, rituxumab, busulfan, capecitabine, pegfilgrastim, mesna, amrubicin, obatoclax, gefitinib, cyclosporine, dasatinib, temozolomide, thiotepa, plerixafor, mitotane, vincristine, doxorubicin, cixutumumab, endostar, fenofibrate, melphalan, sunitinib, rubitecan, enoxaparin, isotretinoin, tariquidar, pomalidomide, sorafenib, altretamine, idarubicin, rapamycin, zevalin, everolimus, pravastatin, carmustine, nelfinavir, streptozocin, tirapazamine, aprepitant, lenalidomide, G-CSF, procarbazine, alemtuzumab, amifostine, valspodar, lomustine, oblimersen, temsirolimus, vinblastine, figitumumab, belinostat, niacinamide, tipifarnib, estramustine, erlotinib, bevacizumab, paclitaxel, docetaxel, cisplatin, carboplatin, Abraxane®, pemetrexed, bortezomib, topotecan, cetuximab, gemcitabine and tetracycline.

12. The combination of claim 11, wherein the one or more therapeutic agents are selected from the list consisting of carboplatin, cisplatin, erlotinib, bevacizumab, bortezomib, paclitaxel, doxorubicin, docetaxel, mitoxantrone, cytarabine and vincristine.

13. A method of treating cancer in a subject, comprising administering to the subject an effective amount of the combination of claim 1.

14. (canceled)

15. The method of claim 13, wherein the cancer is non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, or AID's related Karposi's sarcoma.

16-18. (canceled)

19. The method of claim 15, wherein the cancer is non-Hodgkin's lymphoma.

20. (canceled)

21. The method of claim 19, wherein the non-Hodgkin's lymphoma is a B-cell non-Hodgkin's lymphoma or T-cell non-Hodgkin's lymphoma.

22. The method of claim 21, wherein the B-cell non-Hodgkin's lymphoma is selected from the group consisting of Burkitt's lymphoma, follicular lymphoma, diffuse large B-cell lymphoma, nodal marginal zone B-cell lymphoma, plasma cell neoplasms, small lymphocytic lymphoma/chronic lymphocytic leukemia, mantle cell lymphoma, and lymphoplamacytic lymphoma/Waldenstrom macroglobulinemia.

23. (canceled)

24. The method of claim 21, wherein the T-cell non-Hodgkin's lymphoma is selected from the group consisting of anaplastic large-cell lymphoma, precursor-T-cell lymphoblastic leukemia/lymphoma, unspecified peripheral T-cell lymphoma, and angioimmunoblastic T-cell lymphoma.

25-26. (canceled)

27. A method of inhibiting the growth of a cancer or tumor cell in a subject, the method comprising the steps of: (a) contacting the cell with an effective amount of a compound of formulae (I) or (Ia) as defined in claim 1, and (b) exposing the cell to an effective amount of a topoisomerase II inhibitor, wherein the topoisomerase II inhibitor is selected from the group consisting of etoposide, amsacrine, mitindomide, teniposide, doxorubicin, daunorubicin, idarubicin, mitoxantrone, anteniposide, novobiocin, dexrazoxane, 3-hydroxy-2-[(1R)-6-isopropenyl-3-methyl-cyclohex-2-en-1-yl]-5-pentyl-1,4-benzoquinone, and 4-[2-(3,5-dioxo-1-piperazinyl)-1-methylpropyl]piperazine-2,6-dione.

28. The method of claim 27, wherein the compound is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof and the topoisomerase II inhibitor is etoposide.

29. The method of claim 27, wherein the compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the topoisomerase II inhibitor is etoposide.

30. A method of treating cancer in a subject, comprising administering to the subject an effective amount of the combination of claim 10.

31. The method of claim 30, wherein the cancer is non-small cell lung cancer, extensive small cell lung cancer, osteosarcoma, diffuse large B-cell lymphoma, acute lymphoblastic leukemia, T-cell lymphoblastic lymphoma, acute myelogenous leukemia, anaplastic large cell lymphoma, mantle cell lymphoma, non-Hodgkin's lymphoma, Ewing's sarcoma, pediatric dpendymoma, breast cancer, adenocarcinoma of the prostate, pancreatic cancer, ovarian cancer, hormone refractory prostate cancer, glioblastoma multiforme, gliosarcoma, malignant gliomas, medulloblastoma, adrenocortical cancer, germ cell cancers, refractory testicular cancer, retinoblastoma, anaplastic oligodendroglioma, oligoastrocytoma, peritoneal cancer, fallopian tube cancer, Hodgkin's lymphoma, neuroendocrine carcinoma, hepatocellular cancer, chronic lymphocytic leukemia, chronic myelogenous leukemia, Burkitt's lymphoma, myeloma, or AID's related Karposi's sarcoma.
